(19) United States
(12) Patent Application Publication
(10) Pub. No.: US 2020/0383879 A1
(43) Pub. Date: Dec. 10, 2020

(54) MANIPULATION OF EYELASHES TO REDUCE TEAR EVAPORATION

(71) Applicant: Vance M. Thompson, Sioux Falls, SD (US)

(72) Inventor: Vance M. Thompson, Sioux Falls, SD (US)

(21) Appl. No.: 16/431,095

(22) Filed: Jun. 4, 2019

(51) Int. Cl. A61K 8/02; A61Q 5/06; A41G 5/02; A45D 2/48; A45D 7/02; A45D 40/26

(52) U.S. Cl. CPC A61K 8/027; A61Q 5/06; A45D 40/26; A45D 2/48; A45D 7/02; A41G 5/02

(57) ABSTRACT

A method of mitigating tear evaporation, including manipulating eyelashes to project more downwardly from an upper eyelid, or more upwardly from a lower eyelid or a combination both; and further manipulating at least one of spacing, orientation, direction, contour or shape of the eyelashes to enhance interference with airflow of the eyelashes between a tear film of the eye and an ambient atmosphere to be greater than the interference with airflow which exists because of the eyelashes in an unmanipulated state.

### TECHNICAL FIELD

Embodiments of the invention generally relate to amelioration of dry eye syndrome. More specifically, the invention relates to mitigating tear evaporation.

### BACKGROUND

When refraction of light occurs in the eye the most powerful focusing element of the eye is generally considered to be the anterior surface of the cornea. In fact, focusing primarily occurs at the interface between the tear film and the atmosphere. Accordingly, the tear film is extremely important for refractive reasons in the eye. In addition, the tear film moistens lubricates and assists in nourishing and carrying away metabolic debris from the cornea.

Generally, the tear film is considered to include a mucin layer nearest to the cornea and an aqueous layer overlying the mucin layer and a lipid layer overlying the aqueous layer. There are many circumstances under which the tear film fails to perform adequately and the cornea may suffer because of the tear film's failure to perform properly. For example, in some circumstances the tear film may have a deficiency of the aqueous layer. In further circumstances the mucin layer or lipid layer of the tear film may be insufficient and the tear film may not perform properly. Various deficiencies are improper functions of the tear film are generally categorized as belonging to dry eye syndrome. Many systemic diseases and conditions may also contribute to improper functioning of the tear film.

Dry eye syndrome is one of the most commonly treated eye problems in the United States. Dry eye syndrome is also known as keratitis sicca, keratoconjunctivitis sicca (KCS) xerophthalmia, and lacrimal insufficiency. It is estimated that over ten million Americans and 30 million persons worldwide suffer from dry eye syndrome.

For a large fraction of dry eye patients, dry eye syndrome creates discomfort or annoyance. For those severely afflicted, dry eye syndrome can be debilitating and, in some circumstances, even sight-threatening. In extremely severe cases, dry eye syndrome can even lead to the loss of an eye.

Dry eye syndrome typically results from deficiency in the quality or quantity of tears produced by the patient. Precorneal tear film has traditionally been considered to have a three-layered structure. The closest to the cornea lies the mucin, or mucus, layer. The mucin layer provides an interface between the corneal epithelium and the remainder of the tear film. Overlying the mucin layer is the watery aqueous layer, which is the thickest layer of the three. The outermost layer of the precorneal tear film is the lipid layer. The lipid layer is an oily film that reduces evaporation from the aqueous layer beneath it.

The middle aqueous layer provides moisture to the corneal tissue, carries important nutrients, and serves to remove metabolic waste produced by the cornea. Deficiency in any of the three layers of the precorneal tear film can result in complaints of dry, gritty feeling or burning eyes.

The mucin that forms the mucin layer, nearest the cornea, is secreted by goblet cells in the conjunctiva. The conjunctiva is the transparent tissue that covers the sclera and the backside of the eyelids. The mucin layer functions to decrease surface tension of the tear film. In addition, the cornea itself is hydrophobic. Without the mucin layer to provide a bridge between the cornea and the aqueous layer, the aqueous layer would bead up and allow dry spot formation on the cornea.

The aqueous layer is secreted primarily by the glands of Wolfring and Krause located in the eyelid margin. The aqueous layer helps provide an optically smooth, transparent surface to the precorneal tear film. The lipid layer is secreted by the meibomian glands, and the glands of Zeiss and Moll. The glands of Zeiss and Moll are also located at the eyelid margin.

Blinking is essential to maintenance of the precorneal tear film. During each blink, the eyelid wipes over the surface of the cornea, smoothing the mucin layer and spreading the overlying aqueous and lipid layers to provide a completely wetted surface. In between blinks, the tear film thins due to evaporation of the aqueous layer. If evaporation is excessive, dry spots may form on the surface of the cornea.

The formation of dry spots that may form on the surface of the cornea is sometimes referred to as tear breakup. One clinical test that is utilized to evaluate tear breakup is measuring of the tear breakup time. This is sometimes abbreviated TBUT. To evaluate tear breakup time fluoroscein or a similar dye is introduced to the tears and the tears are observed typically via a slitlamp biomicroscope. If fluoroscein is used the cornea is illuminated with cobalt blue or ultraviolet light. The patient is then asked to hold their eyes open without blinking for as long as possible and the period of time between the last blink and the appearance of dry spots on the cornea is measured and recorded. Tear breakup typically begins at one or several foci on the surface of the cornea and will spread from those foci to form larger dry areas within a short period of time.

Deficiency, or imperfect quality, of any of the three component layers can lead to dry eye symptoms. Many systemic and external factors can contribute to dry eye syndrome. For example, Sjogren's syndrome is associated with arthritic diseases in combination with dry eye and dry mouth. Deficiency of Vitamin A, use of oral contraceptives and environmental factors can all contribute to dry eye syndrome.

Recent research into the natural history of dry eye syndrome has shown that the disease progresses through four stages. Each stage is a consequence of the preceding stage. The stages are:
1. Loss of water from the aqueous layer of the tear film leading to an increase in the tear film osmolarity;
2. Loss of conjunctival goblet cells and decreased corneal glycogen;
3. Increased loss of corneal squamous epithelial cells;
4. Destabilization of the interface between the corneal surface and the tear film.

Either decreased secretion of tear film components or increased evaporation lead to increased tear film osmolarity and the following stages that lead to eventual corneal decompensation and the serious consequences of dry eye syndrome.

The adnexa of the eye may also be involved in dry eye syndrome. The adnexa of the eye include the structures surrounding the eye such as the eyelids, eye lashes, the tear drainage and tear production structures. Blepharitis commonly contributes to dry eye syndrome. Blepharitis typically results from bacterial infection of the tiny glands in the margin of the eyelid. These glands include the glands of Zeiss, Moll and Wolfring as well as the meibomian glands. Most commonly, the affected glands are the meibomian glands. In bacterial blepharitis, bacterial infection causes the meibomian glands to become plugged, and thus not be able to produce a normal lipid layer to contribute to the tear film. Some bacteria that infect the glands also secrete exotoxins that seep out of the glands into the eye and injure the corneal epithelium.

Treatments of dry eye syndrome vary depending upon the type of presentation. The most common treatment for dry eye syndrome is the use of artificial tear supplements to provide additional moisture and lubrication to the corneal surface. Artificial tear eye drops are placed on the eye by the patient. Artificial tear supplements must be used regularly and often to be effective.

Lubricant ointments may also be employed. Ointments are usually used at bedtime because they tend to be messy and blur vision. For some patients, even the use of ointments is not sufficient to provide comfort during sleep.

Tears drain from the eye through the lacrimal drainage system. Tiny openings at the nasal corner of each upper and lower eyelid are called the lacrimal puncta. The lacrimal puncta lead into ducts that drain into the nasopharynx.

One treatment for dry eye syndrome is to partially or completely close one or more lacrimal puncta to reduce tear outflow into the lacrimal drainage apparatus. Traditionally, this closure was accomplished surgically or by cautery. In the last decade, however, temporary and permanent punctal occlusion plugs have been utilized.

Permanent punctal plugs are typically made from surgical silicone; temporary plugs are generally made of soluble collagen. Collagen plugs dissolve over a period of days and are helpful in diagnosis.

Punctal plugs are placed into the lacrimal puncta, or lacrimal drainage ducts. The plugs impede the outflow of tears from the eye. This approach slows the outflow of tears and retains them in the eyes longer, often relieving symptoms. Punctal plugs have the distinct advantage of being readily removable and avoid the issues of scar formation.

Blepharitis is sometimes treated by the use of antibiotic medications. Another important treatment for blepharitis is the application of warm soaks and lid scrubs. In this form of treatment, the patient applies a warm wet washcloth to the eyelids for a period of time to provide humidity, warmth and to help soften blockage of and restore flow from the meibomian glands. Lid scrubs are practiced by taking a mild, nonirritating soap and vigorously scrubbing the eyelid margins with their eyes closed, so as to massage the meibomian glands and increase production.

The surfactant helps to dissolve the greasy blockage of the meibomian glands.

A variety of researchers have been seeking other medicinal treatments for dry eye syndrome. Largely, this research is directed at pharmaceutical efforts to increase tear production.

Recent research has also indicated that inflammation plays a significant role in the development of dry eye syndrome. One treatment based on this research involves the use of anti-inflammatories applied to the eye to mitigate inflammation.

Despite the many treatment options available, there remains no cure for dry eye syndrome. A great many patients still have substantial and even debilitating discomfort because of dry eye syndrome. Very few treatment options exist to provide comfort for dry eye syndrome patients.

### SUMMARY

Existing products and methods that address dry eye syndrome are still insufficient to provide many patients with good comfort, good vision and normal eye health.

Example embodiments of the invention address many of the above problems and concerns.

According to example embodiments, the invention includes methods and devices for manipulating or modifying eyelashes that are expected to mitigate tear evaporation and thus improve the quality and quantity of the tear film for patients having dry eye. Eyelashes, also known as cilia, are an array of hairs located generally at the eyelid margin.

According to the prior art, eyelashes are commonly manipulated for vanity or cosmetic reasons. Various tools, implements and methods are commonly used to curl the eyelashes upwards or to lengthen, thicken and/or separate them to achieve a desired appearance of the eye.

One prior art example of an implement that can be used to manipulate eyelashes is a hand-operated mechanical device such as an eyelash curler. An eyelash curler typically is curved to follow the natural shape of the eyelid and has a lower pad that is pressed against the eyelashes by an opposing narrow bar. Eyelashes are positioned between the lower pad and the upper narrow bar and the tool is closed and the eyelashes are pressed between the lower pad and the upper narrow bar. This curls the eyelashes upwardly on the upper eyelid. Typically the eyelash curler is used to crimp or curl eyelashes upwardly desired resulting in a desirable cosmetic appearance of the eyelashes.

An alternative or adjunct to tools used to manipulate eyelashes is mascara. Mascara is typically applied to eyelashes to increase their appearance of thickness, length and/or separation. This again results in a desired cosmetic appearance.

According to an example embodiment of the invention, eyelashes are manipulated to alter spacing and orientation of the eyelashes to mitigate tear evaporation. According to an example embodiment of the invention, eyelashes are manipulated to decrease exposure of the eye to moving air in the atmosphere. This then, it is expected to tend to mitigate evaporation of the tears.

According to an alternative example embodiment of the invention, further structures or additional materials are applied to the eyelashes to fill in spaces between the eyelashes thus further reducing airflow adjacent the tear film. The materials may, for example, be transparent to maintain a natural appearance of the eye lashes. This is expected to reduce tear film evaporation thus maintaining a moister tear film and to mitigate dry eye symptoms.

According to an example embodiment of the invention, eyelashes are manipulated with different techniques, mechanical tools and methods. A tool according to an example embodiment of the invention may include any of a variety of lash manipulators. For example, tools may include a structure similar to but different from a lash curler or straightener. According to an example embodiment of the invention, a tool can be used to simultaneously bend or straighten lashes while aligning them to achieve a desired effect.

An example embodiment of the invention includes a method of mitigating tear evaporation including manipulating eyelashes to extend more in front of the tear film whether downwardly from the upper eyelid or upwardly from the lower eyelid. The method may further include manipulating at least one of spacing, orientation, direction, contour or shape of the eyelashes to shield the eye and the tear film from the movement of ambient air.

The example method may further include applying a lash setting material to the eyelashes to further hold the eyelashes in a desired orientation and/or to fill in spaces between the eyelashes and/or to thicken the eyelashes.

The example method may further include applying a fibrous substance to the eyelashes to assist in setting the eyelashes. The fibrous material may be applied generally parallel to or generally perpendicular to the normal extension of the natural eyelashes to further mitigate airflow and tear evaporation. In this context, parallel and perpendicular should be broadly interpreted beyond their precise geometric meeting in view of the non-rectilinear nature of the natural eyelashes. For example, the fibrous material may be applied transverse to the eyelashes to facilitate creating a mesh or grid effect to mitigate airflow and thus tear evaporation.

According to another example embodiment, fibrous material may be applied to extend the length of the natural eyelashes to facilitate mitigation of tear evaporation. Fibrous material utilized for extending the length of the eyelashes may include naturally occurring materials such as hairs from the mink or other animals or plant-based materials such as cellulose fibers as well as synthetic materials. Silk may be utilized as well. Any materials that are utilized in the application of cosmetic eyelash extensions can be utilized as well according to example embodiments of the invention.

According to another example aspect of the invention, false eyelashes may be utilized as well to mitigate tear evaporation. False eyelashes differ from eyelash extensions in that they are generally adhered to the eyelid rather than to the eyelashes and in that they are generally made to extend across most of the width of the eyelid. According to example embodiments of the invention, contrary to the known prior art, the false eyelashes extend downwardly from the upper eyelid or upwardly from the lower eyelid in front of the eye in the tear film rather than being curled away from the eye and tear film as is generally the case in cosmetic false eyelashes. The example method may further include inserting micro structures between or applying microstructures to the eyelashes to broaden the eyelashes or to at least partially fill spaces between the eyelashes in an expectation of mitigating tear evaporation.

The example method may further include manipulating spacing between the eyelashes to reduce spacing to shield the eye and the tear film from ambient air movement thereby mitigating tear evaporation. This may include aligning the eyelashes to mitigate or minimize spacing between the eyelashes. This may be done with the upper eyelashes, the lower eyelashes or both.

The example method may further include manipulating the eyelashes with an eyelash manipulating tool.

The example method may further include curling the upper eyelashes downward, curling the lower eyelashes upward or a combination of the foregoing.

The example method may further include making or selecting the eyelash setting material to comprise a wax, an adhesive or a gel.

The example method may further include applying the lash setting material in combination with a fiber material or a fiber grid material. Fiber materials are discussed above. The example method may further include applying heat to the eyelashes to assist in “setting” the lashes in a desired orientation and spacing thereby creating a shielding effect between the ambient air and the tear film and the ocular surface.

According to an example embodiment of the invention, a method of mitigating tear evaporation may include manipulating the eyelashes to position them with an eyelash manipulating tool to cause the eyelashes to extend more downwardly from the upper eyelid, upwardly from the lower eyelid or a combination of the foregoing.

It is expected that eyelashes that are manipulated by curling them downward or straightening them in a downward orientation relative to the upper eyelid will decrease exposure of the eye to ambient air movement and mitigate evaporation of the tear film. Similarly, it is expected that a manipulating eyelashes by curling them upward or straightening them in an upward orientation relative to the lower eyelid will decrease exposure of the eye to ambient atmospheric air movement and mitigate evaporation of the tear film.

The example method may further include application of a substance or substances such as a bioacceptable eyelash setting product, an eyelash grid material or substance and/or micro structures to shield the eye from ambient air movement and thus mitigate tear evaporation. According to an example embodiment, application of a substance or substances can be made with or without use of an eyelash manipulating tool.

According to a further example embodiment of the invention, a bio acceptable eyelash setting material can include for example, a wax, adhesive or gel that can assist in setting the eyelashes to keep them in a manipulated orientation or state. Such material can be applied utilizing an eyelash brush or any other brush or applicator suitable for application of the product to eyelashes. In some aspects of the invention, the lash setting product is applied to the eyelashes after an eyelash manipulating tool has been utilized. In further aspects of the invention, the lash setting material can be applied to bare eyelashes. According to a further aspect of an example embodiment of the invention, the lash setting material can be applied to the eyelashes prior to manipulation of the eyelashes.

According to a further example embodiment of the invention and eyelash grid material or structure may be applied to the eyelashes. According to some aspects, this may include a bioacceptable material that contains fibers or structures. The lash grid material can be applied to bare eyelashes or to eyelashes precoated with a lash setting material. A fibrous component may be utilized to thicken the eyelashes or to fill spaces between the eyelashes.

According to a further example embodiment, application of the fibrous material to the eyelashes can create a gridlike structure on or adjacent to the eyelashes. According to a further example embodiment a grid, mesh or screen may be applied to the eyelashes to mitigate ambient air flow near the ocular surface and/or tear film to mitigate evaporation of the tear film.

According to a further example embodiment, artificial or simulated eyelashes may be placed in between the natural eyelashes and in an example embodiment supported by bridges between the natural eyelashes.

According to another example embodiment of the invention, micro-structures may be applied to or between the natural eyelashes to shield the ocular surface and tear film from ambient airflow.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of a prior art tool;

FIG. 2 is a perspective view of another prior art tool;

FIG. 3 is a perspective view of a further prior art tool;

FIG. 4 is a perspective schematic view of an eye and eyelashes in their unmanipulated orientation;

FIG. 5 is a cross-sectional schematic view of an eyelid and eyelashes in their unmanipulated orientation;

FIG. 6 is a perspective schematic view of an eye and lie lashes having been manipulated in accordance with an example embodiment of the invention;

FIG. 7 is a cross-sectional schematic view of an eyelid and eyelashes having been manipulated according to an example embodiment of the invention;

FIG. 8 is a flow chart of a method according to an example embodiment of the invention;

FIG. 9 is a schematic depiction of a grid structure according to an example embodiment of the invention;

FIG. 10 is a schematic depiction of a grid structure with defined apertures located within the grid according to an example embodiment of the invention;

FIG. 11 depicts a series of horizontal fibers applied across the natural eyelashes according to an example embodiment of the invention;

FIG. 12 depicts a series of micro-structures placed between the natural eyelashes according to an example embodiment of the invention;

FIG. 13 depicts artificial eyelashes inserted between the natural eyelashes and supported in place by bridging structures according to an example embodiment of the invention, and

FIG. 14 is a perspective view of an eyelash manipulator tool according to an example embodiment of the invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

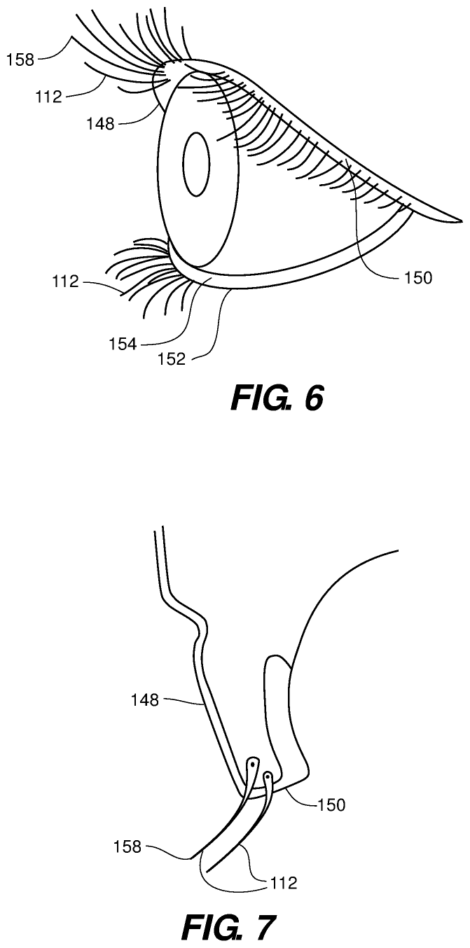

Referring, for example, to FIGS. 6, 7, 11 and 12, according to an example embodiment of the invention, tear evaporation is expected to be mitigated by manipulation of eyelashes 100.

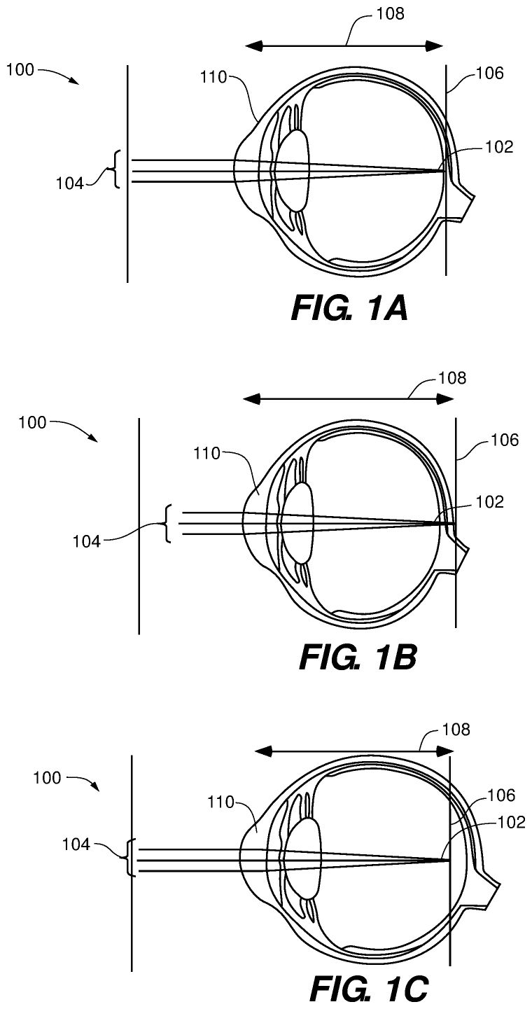

Referring to FIGS. 1-3, eyelash manipulators 102 including eyelash curlers, and various brushes and combs are known in the art, but the prior art structures, so far as applicant is aware, are uniformly intended and generally applied to curl upper eyelashes 100 upward for aesthetic appeal.

Referring to FIG. 1, prior art eyelash manipulator 102 includes lash separating structures 104 such as straight or curved metal comb 106 and handle 108. Handle 108 may optionally be foldable for ease of storage.

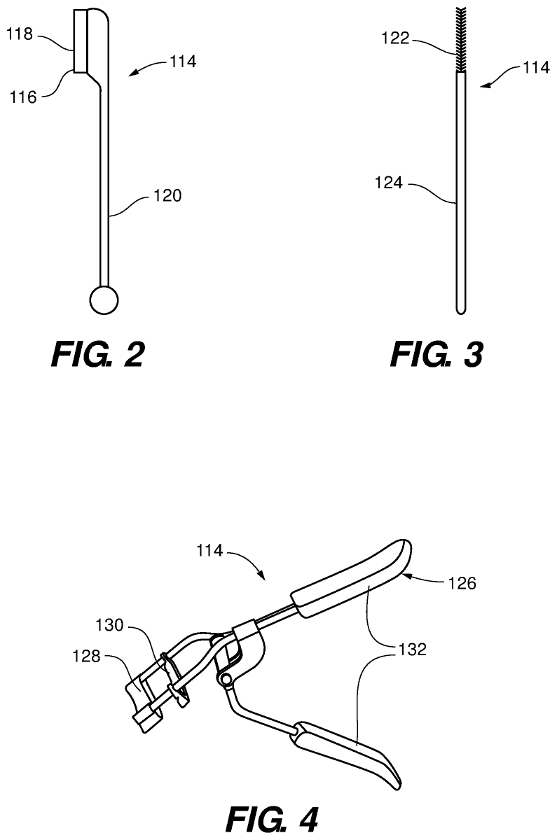

Referring to FIG. 2, prior art eyelash manipulator 102 may alternatively include fibrous bristles 110 and straight handle 112. Compared to the rigid and straight lash separating of metal comb 106 lash separating structure 104 has a spoolie shape and is softer on eyelashes 100.

Referring now to FIG. 3 according to the prior art, eyelash curler 114 can be used to manipulate eyelashes and curl them upwards from the upper eyelid for aesthetic appearance. Generally eyelash curler 114 includes upper rigid curved top 116 and lower curved resilient padded base 118 as well as mutually opposed handles 120. In application, eyelashes 100 are positioned between upper rigid curved top 116 and lower curved resilient padded base 118 while opposed handles 120 are opened to admit eyelashes 100. Opposed handles 120 are then closed similar to a pair of scissors. Closing of opposed handles 120 forces upper rigid curved top 116 and resilient padded base 118 together, thereby crimping and curling eyelashes 100 upwardly to create a desired aesthetic appearance.

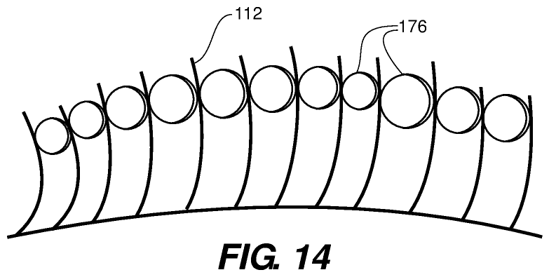

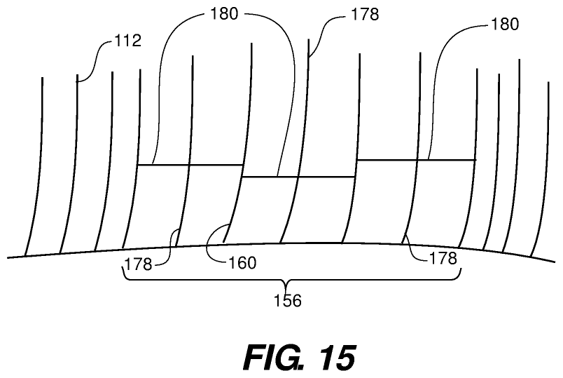

Referring now to FIG. 14, eyelash manipulator 122 according to an example embodiment of the invention, includes handles 124, upper curved resilient padded base 126, curved bar 128 and optionally, removable toothed insert 130. Eyelash manipulator 122 may further include heating element 131.

In the depicted embodiment, curved bar 128 is slidably movable between a first position located away from upper curved resilient padded base 126 and a second position in contact with upper curved resilient padded base 126. Optional toothed insert 130 may be coupled to either upper curved resilient padded base 126 or curved bar 128. Teeth 132 of removable toothed insert 130 may be sized and spaced to facilitate spacing eyelashes 100 closer together. Removable toothed insert 130 and/or upper curve resilient padded base 126 may further include reservoir 134 adapted to contain lash setting material. When curved bar 128 contacts upper curved resilient padded base 126, curved bar 128 is impressed into upper padded base 126 because of the resiliency thereof. Handles 124 are linked to curved bar 128 and upper curved resilient padded base 126 to accomplish slidable or other relative movement of the two parts relative to one another.

Referring to FIG. 4, in the schematic depiction, under normal circumstances eyelashes 100 of upper eyelid 136 extend outwardly and upwardly from upper eyelid margin 138. Eyelashes 100 of lower eyelid 140 extend generally outwardly and downwardly from lower eyelid margin 142. While example embodiments of the invention presented in this application generally refer to upper eyelashes 148, it should be understood that the invention may be applied lower eyelashes 149 as well so that upper eyelashes 100 may be manipulated, the lower eyelashes 149 may be manipulated or both may be manipulated.

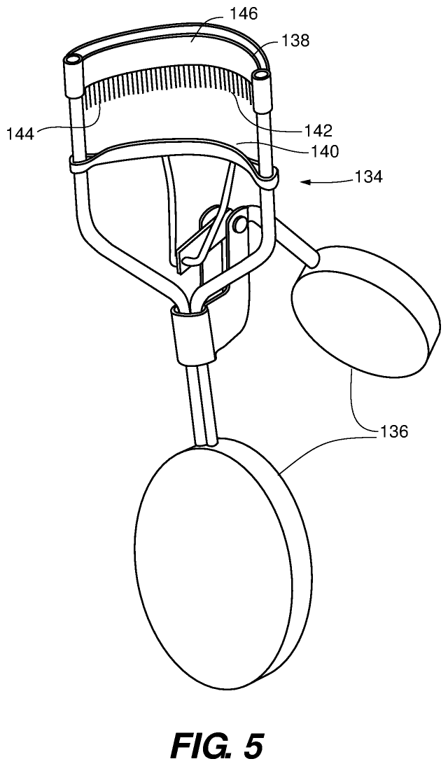

Referring to FIG. 5, in a cross-sectional view of upper eyelid 136 it can be seen that, under normal circumstances, upper eyelid margin 138 is a roughly planar structure from which eyelashes 100 of upper eyelid 136 extend. While eyelashes 100 exit upper eyelid margin 138 in a generally downward and outward direction, the normal curvature of eyelashes 100 in addition to the structure of upper eyelid 136 generally causes eyelashes 100, in particular eyelashes 100 of central portion 144 to extend outwardly and upwardly at their distal terminus 146.

Referring to FIG. 6, according to an example embodiment of the invention at least central upper eyelashes 148 of upper eyelid 136 extend downwardly from upper eyelid 136 thus partially shielding eye 150 from ambient air.

Referring now to FIG. 7, central upper eyelashes 148 are depicted after having been manipulated according to an example embodiment of the invention, to extend downwardly from upper eyelid 136.

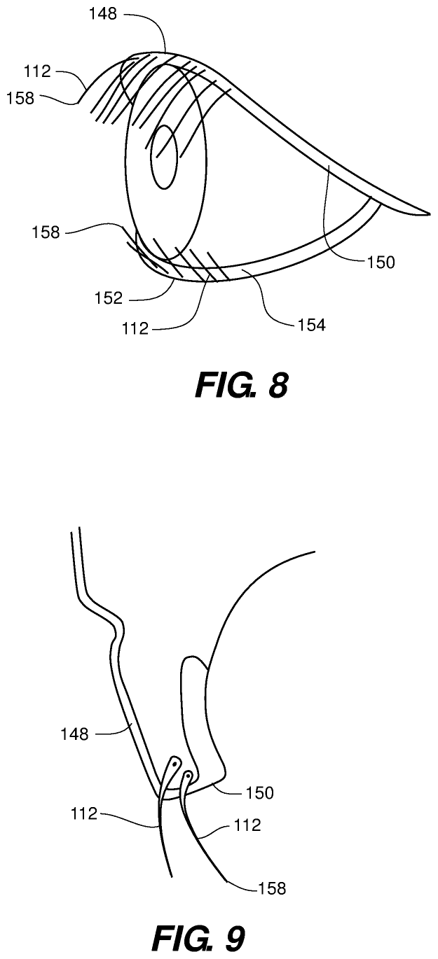

According to a further example embodiment of the invention, with reference to FIG. 8, a method of mitigating tear evaporation generally includes manipulating eyelashes 100 to project more downwardly from an upper eyelid 136 or more upwardly from a lower eyelid 140 or a combination of both. S1A And, further manipulating at least one of spacing, orientation, direction, contour or shape of eyelashes 100 to increase a barrier between a tear of eye 100 and an ambient atmosphere to a greater degree than that which exists because of eyelashes 100 in an unmanipulated state. S1 B.

According to a further example embodiment of the invention the method further includes applying a lash setting material to eyelashes 100. S2. Eyelashes may be adhered, joined or fused together with the lash setting material to mitigate tear evaporation.

According to a further example embodiment, the method further includes applying a fibrous substance to eyelashes 100 to assist in setting the eyelashes to mitigate tear evaporation S3.

The fibrous material may be applied generally parallel to or generally perpendicular to the normal extension of natural eyelashes 100. In this context, parallel and perpendicular should be broadly interpreted beyond their precise geometric meeting in view of the non-rectilinear linear nature of natural eyelashes 100. For example, the fibrous material may be applied transverse to eyelashes 100 to facilitate mitigation of tear evaporation. According to another example embodiment, fibrous material may be applied to extend the length of the eyelashes to facilitate extension to facilitate mitigation of tear evaporation. Fibrous material utilized for extending the length of eyelashes 100 may include naturally occurring materials such as hairs from the mink or other animals or plant-based materials such as cellulose fibers as well as synthetic materials. Silk may be utilized as well. Any materials that are utilized in the application of cosmetic eyelash extensions can be utilized as well according to example embodiments of the invention.

According to another example aspect of the invention, false eyelashes 166 may be utilized as well. False eyelashes 166 differ from eyelash extensions in that they are generally adhered to upper eyelid 136 rather than to eyelashes 100 and that they are generally made to extend across most of the width of upper eyelid 136. According to example embodiments of the invention, contrary to the known prior art, the false eyelashes 166 extend downwardly from upper eyelid 136 or upwardly from lower eyelid 140 to shield the tear film and mitigate tear evaporation rather than being curled as in cosmetic false eyelashes 166.

According to a further example embodiment, the method further includes inserting micro-structures 164 between or applying micro-structures to eyelashes 100. S4.

According to another example embodiment, the method further includes manipulating spacing between eyelashes 100 to reduce spacing between eyelashes 100 to mitigate tear evaporation. S5.

According to another example embodiment, the method further includes applying heat to the eyelashes 100. S6.

According to a further example embodiment, the method further includes applying simulated eyelashes 166 in between eyelashes 100 along with bridges 168 supporting simulated eyelashes 166 between eyelashes 100 to at least partially fill spaces between the eyelashes 100.

S7.

According to another example embodiment of the invention, the method further includes manipulating eyelashes 100 with a eyelash manipulator 122. S8.

According to another example embodiment of the invention, the method further includes curling upper eyelashes 148 downward. S9.

According to another example embodiment of the invention, the method further includes applying grid structure 152 to upper eyelashes 148 to mitigate air passage through eyelashes 100 to mitigate tear evaporation. S10

According to another example embodiment of the invention, the method further includes making or selecting eyelash setting material to comprise a wax, an adhesive or a gel. S11.

According to another example embodiment of the invention, the method further includes applying the lash setting material in combination with fiber material or fiber grid material. S12.

According to another example embodiment of the invention, the method further includes applying heat to eyelashes 100 to facilitate setting in a desired position. S13.

Referring to FIG. 9, grid structure 152, according to an example embodiment of the invention is depicted. Grid structure 152 may be applied to upper eyelashes 148 or lower eyelashes 149 thereby further facilitating mitigation of tear evaporation. Grid structure 152 may include, for example, horizontal grid members 154 and vertical grid members 156. Grid structure 152 may be held in reservoir 134 along with or separate from lash setting compound.

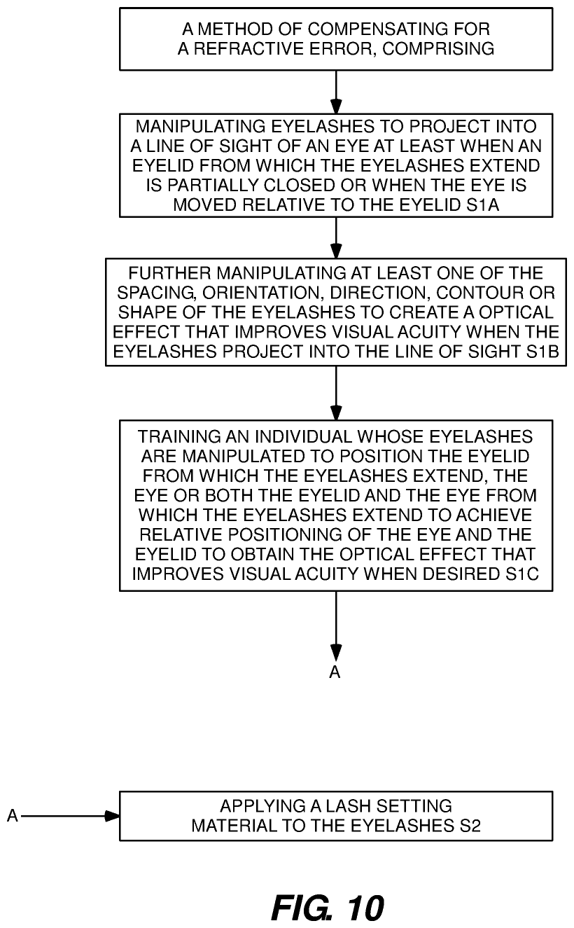

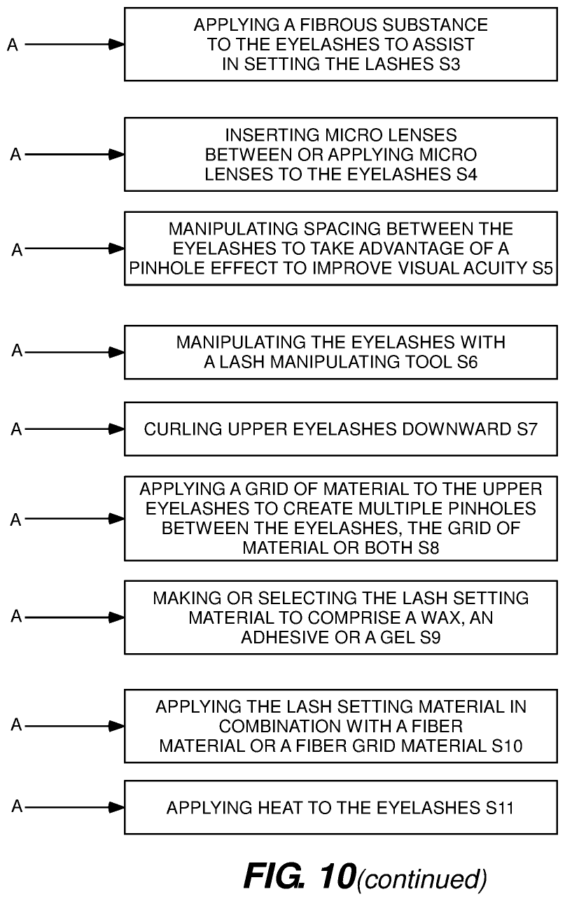

Referring to FIG. 10, grid structure 152 according to an example embodiment of the invention may further include aperture structures 158. Aperture structures 158 may for example include structures defining a central circular aperture 160. Central circular apertures 160 may be of a consistent size or may be of variable sizes according to example embodiments of the invention. Grid structure 152 with aperture structures 158 may be held in reservoir 134 along with or separate from lash setting compound.

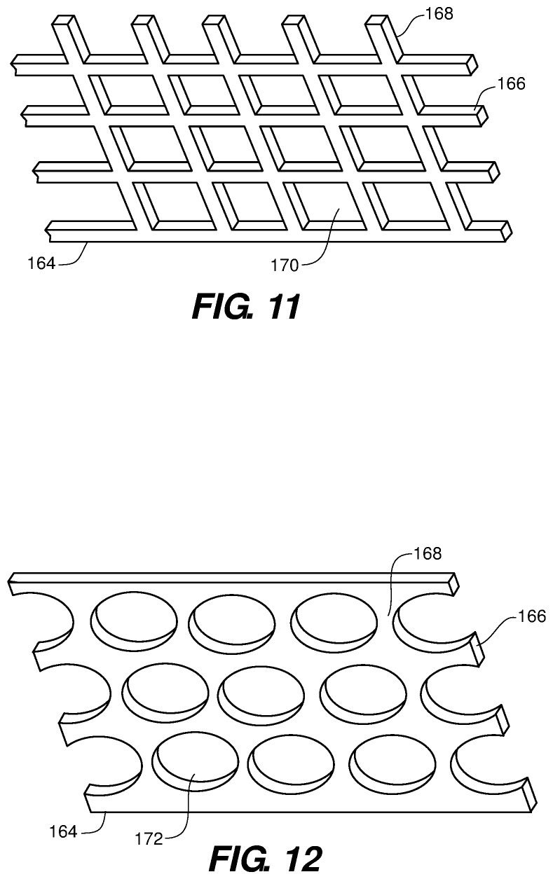

Referring to FIG. 11, a series of horizontal fibers 162 are depicted as applied across upper eyelashes 148 according to an example embodiment of the invention. Horizontal fibers 162 may be secured to natural upper eyelashes 148 by a lash setting compound as discussed elsewhere in this application. Horizontal fibers 162 may be held in reservoir 134 along with or separate from lash setting compound.

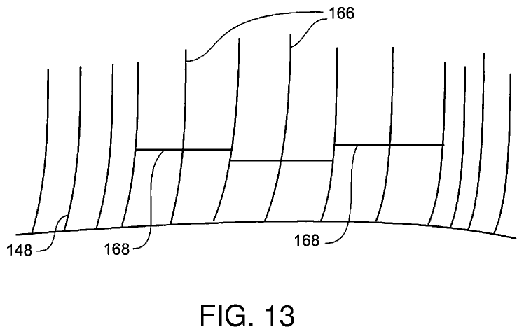

Referring to FIG. 12 according to an example embodiment of the invention, micro-structures 164 may be inserted on between or adjacent to eyelashes 100. Micro-structures 164 may vary in size and density and may be secured to or between eyelashes 100. Micro-structures 164 may be held in reservoir 134 along with or separate from lash setting compound.

Referring to FIG. 13, according to another example embodiment of the invention, two or more false or simulated eyelashes 166 may be inserted adjacent to or between upper eyelashes 148 and supported by bridges 168 inserted between natural central upper eyelashes 100.

Similarly, two or more false or simulated eyelashes 166 may be inserted adjacent to or between lower eyelashes 149. Accordingly, desired reduction in spacing between a combination of false or simulated eyelashes 166 and eyelashes 100 are created. Bridges 168 may be inserted between eyelashes 100 for example of the upper eyelid 136 as well as eyelashes 100 peripherally located or of lower eyelid 140. Further, false or simulated eyelashes 178 may either envelop eyelashes 100 or be located between eyelashes 100 to facilitate mitigation of tear evaporation. Further, false or simulated eyelashes 178 as well as bridges 168 if utilized may be held in reservoir 134 along with or separate from lash setting compound.

In operation, referring to FIG. 14, eyelash manipulator 122 is positioned over upper eyelashes 148 so that upper eyelashes 148 are located between upper curve resilient padded base 126 and curved bar 128. Handles 124 are brought together to impress curved bar 128 into upper curve resilient padded base 126. If present, heating element 131 is activated to apply heat to upper eyelashes 148 to assist in curving them downward.

If utilized, grid structures 152, horizontal fibers 162, microstructures 164, false or simulated eyelashes 166 and/or bridges 168 are applied to upper eyelashes 148. Lash setting compound is applied to facilitate adhesion of the foregoing to upper eyelashes 148.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method of mitigating tear evaporation, comprising:
manipulating eyelashes to project more downwardly from an upper eyelid, or more upwardly from a lower eyelid or a combination both; and
further manipulating at least one of spacing, orientation, direction, contour or shape of the eyelashes to enhance interference with airflow of the eyelashes between a tear film of the eye and an ambient atmosphere to be greater than the interference with airflow which exists because of the eyelashes in an unmanipulated state thereby mitigating tear evaporation.

2. The method as claimed in claim 1, further comprising applying a lash setting material to the eyelashes.

3. The method as claimed in claim 1, further comprising applying a fibrous substance to the eyelashes to assist in setting the lashes to mitigate tear evaporation.

4. The method as claimed in claim 1, further comprising inserting micro-structures between or applying micro-structures to the eyelashes thereby at least partially filling spaces between the eyelashes.

5. The method as claimed in claim 1, further comprising manipulating spacing between the eyelashes to reduce spacing between the eyelashes to mitigate tear evaporation.

6. The method as claimed in claim 1, further comprising manipulating spacing between the eyelashes to make the eyelashes closer together.

7. The method as claimed in claim 1, further comprising manipulating the eyelashes with an eyelash manipulator.

8. The method as claimed in claim 1, further comprising curling upper eyelashes downward from a upper eyelid, curling lower eyelashes upward from a lower eyelid or a combination of both of the foregoing.

9. The method as claimed in claim 1, further comprising applying a grid of material to the eyelashes to at least partially fill spaces between the eyelashes.

10. The method as claimed in claim 2, further comprising making or selecting the lash setting material to comprise a wax, an adhesive or a gel.

11. The method as claimed in claim 10, further comprising applying the lash setting material in combination with a fiber material or a fiber grid material applied to the eyelashes.

12. The method as claimed in claim 1, further comprising applying heat to the eyelashes.

13. The method as claimed in claim 1, further comprising applying simulated eyelashes in between the eyelashes along with bridging structures supporting the simulated eyelashes between than the eyelashes to at least partially fill spaces between the eyelashes.

14. The method as claimed in claim 9, further comprising selecting or making the grid of material to reduce spaces between the eyelashes.

15. A tool for manipulating upper eyelashes, comprising:
an upper resilient member and a lower rigid member, the lower rigid member being narrower than and resiliently receivable into the upper resilient member by indentation of the upper resilient member;
wherein the upper resilient member and the lower rigid member are movable relative to one another from a first position in which the upper resilient member and the lower rigid member are separated by a distance to a second position in which the lower rigid member impinges upon the upper resilient member and compresses a central portion of the upper resilient member thereby facilitating curving eyelashes of an upper eyelid located therebetween into a downward orientation; and
a reservoir containing a lash setting material and positioned to transfer the lash setting material to the eyelashes.

16. The tool as claimed in claim 15, further comprising a second reservoir containing fibrous material to be applied with the lash setting material to facilitate reducing spaces between the eyelashes.

17. The tool as claimed in claim 15, further comprising a second reservoir containing mesh material to be applied with the lash setting material to facilitate reducing spaces between the eyelashes.

18. The tool as claimed in claim 15, further comprising a heating element located to apply heat to the eyelashes.

19. The tool as claimed in claim 15, further comprising a second reservoir containing microstructures to be applied to or between the eyelashes to at least partially fill spaces therebetween.

20. The tool as claimed in claim 15, further comprising a second reservoir containing simulated lashes, bridges or both to be applied to and between the eyelashes to at least partially fill spaces therebetween.
